SEQUENCE LISTING (35 sequences)

SEQ ID NO: 1 - DNA, 27 bp, Artificial Sequence
  Primer
gacggatccatggtccaagcccatggg

SEQ ID NO: 2 - DNA, 27 bp, Artificial Sequence
  Primer
caggtcgactcaggggctgggttcctc

SEQ ID NO: 3 - DNA, 25 bp, Artificial Sequence
  Primer
ggcctggccagcccatcagacacca

SEQ ID NO: 4 - DNA, 29 bp, Artificial Sequence
  Primer
aggggggatgatcgcgacggcagagcagt

SEQ ID NO: 5 - DNA, 27 bp, Artificial Sequence
  Primer
gacggatccatgggagcctccttctgg

SEQ ID NO: 6 - DNA, 28 bp, Artificial Sequence
  Primer
caggtcgactcaggggctgggttcctca

SEQ ID NO: 7 - DNA, 25 bp, Artificial Sequence
  Primer
ggcctggccagcccatcagacacca

SEQ ID NO: 8 - DNA, 29 bp, Artificial Sequence
  Primer
aggggggatgatcgcgacggcagagcagt

SEQ ID NO: 9 - DNA, 26 bp, Artificial Sequence
  Primer
atagaattcatggagcccgcgaccgc

SEQ ID NO: 10 - DNA, 30 bp, Artificial Sequence
  Primer
atatctagattactgactctccacggccag

SEQ ID NO: 11 - DNA, 29 bp, Artificial Sequence
  Primer
ttgagctggccagcctatggtgtcccttc

SEQ ID NO: 12 - DNA, 29 bp, Artificial Sequence
  Primer
ggccttcagcatcgcgacccctgagcagt

SEQ ID NO: 13 - DNA, 30 bp, Artificial Sequence
  Primer
gacgaattcatgacagccatcatcaaagag

SEQ ID NO: 14 - DNA, 34 bp, Artificial Sequence
  Primer
cagtctagatcagacttttgtaatttgtgtatgc

SEQ ID NO: 15 - DNA, 35 bp, Artificial Sequence
  Primer
cacaatatccttttgaagcccataacccaccacag

SEQ ID NO: 16 - DNA, 39 bp, Artificial Sequence
  Primer
tcatgttgcagcaattcacgctaaagctggaaagggacg

SEQ ID NO: 17 - DNA, 27 bp, Artificial Sequence
  Primer
gacgaattcatgacatcgcggagatgg

SEQ ID NO: 18 - DNA, 33 bp, Artificial Sequence
  Primer
cagtctagatcatctgaaacttttctgctgttg

SEQ ID NO: 19 - DNA, 23 bp, Artificial Sequence
  Primer
gacctggccggcccacggcgtgc

SEQ ID NO: 20 - DNA, 43 bp, Artificial Sequence
  Primer
ccagcattatattgaaacactagcgcgcaggattgaagaagag

SEQ ID NO: 21 - DNA, 27 bp, Artificial Sequence
  Primer
gacgaattcatgacatcgcggagatgg

SEQ ID NO: 22 - DNA, 27 bp, Artificial Sequence
  Primer
cagtctagatcacctgcagtgcaccac

SEQ ID NO: 23 - DNA, 23 bp, Artificial Sequence
  Primer
gacctggccggcccacggcgtgc

SEQ ID NO: 24 - DNA, 27 bp, Artificial Sequence
  Primer
gacgaattcatgcccaccaccatcgag

SEQ ID NO: 25 - DNA, 32 bp, Artificial Sequence
  Primer
caggtcgacattgtttatagggcattttgctg

SEQ ID NO: 26 - DNA, 35 bp, Artificial Sequence
  Primer
ttatactacctggccagcttttggagtccctgaat

SEQ ID NO: 27 - DNA, 37 bp, Artificial Sequence
  Primer
accgaatgggtcttattgcgaccccagatcaactgag

SEQ ID NO: 28 - DNA, 30 bp, Artificial Sequence
  Primer
gacggatccatggagcaagtcgagatcctg

SEQ ID NO: 29 - DNA, 32 bp, Artificial Sequence
  Primer
caggtcgactcattcaacaaggcaactgcggg

SEQ ID NO: 30 - DNA, 36 bp, Artificial Sequence
  Primer
tatgtgaactggccvagcccatgatgttccttcatc

SEQ ID NO: 31 - DNA, 43 bp, Artificial Sequence
  Primer
cacaaaggcattctgcagtagcaacaaaggagcaatatgaact

SEQ ID NO: 32 - DNA, 31 bp, Artificial Sequence
  Primer
gacggatccatggagatggaaaaggagttcg

SEQ ID NO: 33 - DNA, 31 bp, Artificial Sequence
  Primer
caggtcgacctatgtgttgctgttgaacagg

SEQ ID NO: 34 - DNA, 29 bp, Artificial Sequence
  Primer
taccacatggcctgcctttggagtccctg

SEQ ID NO: 35 - DNA, 29 bp, Artificial Sequence
  Primer
gatggggctgatcgcgacagccgaccagc

(19) United States
(12) Patent Application Publication
(10) Pub. No.: US 2006/0127965 A1
(43) Pub. Date: Jun. 15, 2006

(54) Bindingzyme arrays and high-throughput proteomic methods

(76) Inventor: Charlie Rodi, Del Mar, CA (US)

Correspondence Address: MORRISON & FOERSTER LLP, 12531 HIGH BLUFF DRIVE, SUITE 100, SAN DIEGO, CA, 92130-2040, US

(21) Appl. No.: 10/750,353

(22) Filed: Dec. 31, 2003

Related U.S. Application Data
(60) Provisional application No. 60/437,221, filed on Dec. 31, 2002.

(51) Int. Cl. C12Q 1/37; G01N 33/00

(52) U.S. Cl. 435/23; 436/86

(57) ABSTRACT

Provided herein are methods for identifying the presence or absence of a polypeptide variance between different biological samples and corresponding methods for generating a high-throughput screen to rapidly identify variances of one or more polypeptides in different biological samples. In particular, a variance in a post-translational modification on a particular polypeptide in the biological samples can be identified, such as the presence or absence of a polypeptide having an attached phosphoryl moiety, for example. In these methods, a catalytically inactivated enzyme (i.e. bindingzyme) is utilized as a substrate-specific binding protein. These bindingzymes can bind to one or more substrates in biological samples and a bound substrate can act as a marker to distinguish one sample from another. These methods also are useful for isolating substrates for their identification, for the detection of substrates in a sample, and for the discovery and development of ethical drugs.

### RELATED PATENT APPLICATIONS

This patent application claims the benefit of provisional patent application No. 60/437,221 filed Dec. 31, 2002, entitled “Bindingzyme arrays and high-throughput proteomic methods,” naming Charlie Rodi as an inventor and having attorney docket number 532873000100. This application is hereby incorporated herein by reference in its entirety, including all drawings, cited publications and documents.

### FIELD OF THE INVENTION

The invention is directed to the field of proteomics, which generally involves identifying polypeptide variances among different biological samples. In particular, the invention is directed to methods and corresponding components for carrying out high-throughput screens that identify polypeptide variances among biological samples.

### BACKGROUND

In the field of proteomics polypeptide variances often are distinguished by two-dimensional gel (2D gels) analyses (Freed & Hunter (1992) Mol. Cell. Biol. 12: 1312-1323) or newer mass-spectrometry-based methods (Conrads et al. (2002) BBRC 290: 885-890). By way of example, some proteomics approaches have focused upon variances of phosphorylated proteins (phosphoproteins).

Phosphoproteins are important components of signal transduction processes that regulate cell cycle control, differentiation, response to growth factors, and other cellular phenomena. Changes in many critical signal transduction events can be tracked using certain research techniques that analyze phosphoproteins. For instance, researchers can add radioactive ³²P to two different cell cultures, extract the proteins, and then separate them on 2D gels. By exposing each gel to X-ray film, the researchers can view the pattern of radioactively labeled phosphoproteins characteristic for each sample. A comparison of the patterns can reveal changes in the phosphoprotein content of the two samples. Not only do these phosphoprotein changes represent functional differences between the two cell states, but they could also be used as markers in a cell-based screen of a chemical compound library for small molecules that can either mimic the changes or reverse them, depending on need. Though a valuable research tool, 2D gels are imprecise, complicated to analyze, cumbersome, labor-intensive, use radioactive phosphorous and have a poor dynamic range. Each of these drawbacks compromise their utility in making comprehensive phosphoprotein comparisons between samples and they are unsuited for high-throughput screening.

Some improvements in the comprehensive analysis of phosphoproteins have been made, but they too have their problems. Certain methods separate the phosphoproteins from non-phosphorylated proteins for subsequent analysis by mass spectrometry (MS). Although the dynamic range of MS is superior to 2D gels, peak suppression can still cause data to be missed. Peak suppression is a phenomenon in which a dominating peak (or peaks) suppresses the signal from less prominent peaks. Peak suppression can be compounded by the fact that phosphoprotein peaks also are often suppressed in MS to begin with. Although current phosphoprotein enrichment techniques reduce the complexity of a sample, that reduction is not by much, since as much as 30% of the proteins in a sample may be phosphorylated. Also, current methods include many processing steps, which can consist of chemical treatments, chromatography, repeated washing, and elution, leading to reduced yields and questions of reproducibility from sample to sample. Taken together, these shortcomings make these methods incompatible with high-throughput screens.

The characteristics described above in connection with phosphoproteins also exist for other classes of proteins modified by post-translational modification processes, including but not limited to ubiquitinated proteins, acetylated proteins, myristoylated proteins, and methylated proteins.

### SUMMARY

The methods and components described hereafter allow for a rapid and reliable identification of a polypeptide variance in a comparison of two or more biological samples. Such methods and components allow for rational and simple development of an assay compatible with high-throughput screening.

Thus, provided herein are methods for identifying the presence or absence of a polypeptide variance between two biological samples, which comprise contacting a first biological sample with an inactivated enzyme in a first system and contacting a second biological sample with the inactivated enzyme in a second system. The inactivated enzyme is capable of binding to a native polypeptide substrate or a fragment thereof and is catalytically defective, and these inactivated enzymes are referred to herein as “bindingzymes.” A bindingzyme often is capable of binding to a binding site on the native polypeptide substrate or fragment thereof that comprises a modification capable of being added to the polypeptide by a native post-translational modification process. In specific embodiments, the bindingzyme is an inactivated phosphatase that binds to phosphorylated polypeptides or fragments thereof. A signal corresponding to a polypeptide bound to the inactivated enzyme is then detected in the first system and in the second system, and the signals in the two systems are compared. A difference between the signals identifies the presence of a polypeptide variance between the first biological sample and the second biological sample and the absence of a difference between the signals identifies the absence of a polypeptide variance.

In specific embodiments, bindingzymes identified as being informative (e.g., useful for detecting a signal difference between different biological samples) or non-informative (e.g., do not detect a signal difference between biological samples) in the methods described above are selected independently and utilized in subsequent screens similar to those described above. These subsequent screens often further comprise administering test molecules to each biological sample and comparing signals generated in each system. Such screens can be used to identify modulators of biological processes and to assess modulator toxicity and specificity.

Thus, provided are methods for identifying a molecule that reduces a polypeptide variance between two biological samples, which comprise contacting a first biological sample with one or more inactivated enzymes in a first system and contacting a second biological sample with the one or more inactivated enzymes and one or more test molecules in a second system. The one or more inactivated enzymes often are capable of binding to a native polypeptide substrate or a fragment thereof and often are catalytically defective. Also, one or more of the inactivated enzymes often are capable of detecting the presence of a polypeptide variance between the two biological samples. A signal corresponding to a polypeptide bound to the one or more inactivated enzymes in the second system often is detected and compared with a corresponding signal in the first system. A test molecule that reduces the difference between the signals relative to the difference between the signals in the absence of the test compound often is identified as a molecule that modulates a polypeptide variance between two biological samples.

Also provided are methods for constructing an array of inactivated enzymes, which comprise contacting a first biological sample with inactivated enzymes in a first system and contacting a second biological sample with the inactivated enzymes in a second system. The inactivated enzymes often are capable of binding to a native polypeptide substrate or a fragment thereof and often are catalytically defective. Signals corresponding to polypeptides bound to the inactivated enzymes in the first system and signals corresponding to polypeptides bound to the inactivated enzymes in the second system often are detected and compared. Inactivated enzymes for which there is a difference between a signal in the first system and a signal in the second system often are identified as informative inactivated enzymes and inactivated enzymes for which there is no detectable difference between the signals often are identified as uninformative inactivated enzymes. One or more of the informative inactivated enzymes often are deposited in an array and sometimes one or more uninformative inactivated enzymes are deposited in the array. In certain embodiments, uninformative inactivated enzymes are deposited in an array other than an array containing informative bindingzymes (e.g. arrays containing non-informative bindingzymes can be utilized to determine selectivity of a test molecule and as internal standards, for example, as described in greater detail below).

Also provided is an array comprising two or more inactivated enzymes immobilized to a solid support, where each inactivated enzyme is capable of binding to a native polypeptide substrate or a fragment thereof and is catalytically defective. In certain embodiments, one or more inactivated enzymes in the array are identified by methods described herein as being capable of detecting the presence of a polypeptide variance between two biological samples. The array often includes different bindingzymes having different binding profiles, such as bindingzymes illustrated in FIG. 4A. Also provided is a system comprising an array of bindingzymes described herein and a mass spectrometer.

### BRIEF DESCRIPTION OF THE DRAWINGS

FIG. 1 contrasts a bindingzyme from a catalytically active enzyme. In the left panel, the catalytically active phosphatase is shown bound to a solid support. The phosphatase binds its substrate (a phosphorylated polypeptide), catalytically removes the phosphate group, then releases both the polypeptide and the phosphate moieties. In the right panel, the bindingzyme is shown bound to a solid support. Though catalytically inactive (e.g., due to substitution of a serine residue for a cysteine residue in the active site), the bindingzyme retains the specific binding activity of the phosphatase from which it was derived. It binds and retains its substrate without altering it, and the substrate thereby is more stably bound by the bindingzyme than it would be by the phosphatase.

FIG. 2 illustrates particular embodiments for constructing and using a panel of bindingzymes to determine polypeptide variances between two samples, and a subsequent configuration of informative bindingzymes into high-throughput screening assays. In this illustration, polypeptide variances arising from treatment of cultured cells with erythropoietin (EPO) are detected. In part A, two identical cell cultures are used, except that the Control Culture is not treated with EPO, whereas the Experimental Culture is treated with EPO. The cells are lysed, and may be treated or untreated with endoproteases, then with protease inhibitors. In part B, an aliquot of the Control lysate is added to each well of a multiwell plate (MWP), with each well containing a different bindingzyme tethered to it (each derived from a different phosphatase), and an aliquot of the Experimental lysate is added to each well of an identical MWP. After allowing time for binding to take place, each well is washed to remove unbound material. In part C, retained material is eluted and examined by MALDI-TOF MS. No polypeptide variance is seen for bindingzymes in the non-darkened wells, demonstrating that they are non-informative bindingzyme. The bindingzyme in the darkened well, however, does detect polypeptide variances between the two samples, making it an informative bindingzyme. Part D depicts a complete comparison of all 96 different bindingzymes used in this illustration, where the 90 non-darkened wells represent non-informative bindingzymes, and the 6 darkened wells represent informative bindingzymes (for the two samples examined). In part E, each well contains one or more of the 6 informative bindingzymes. Identical MWPs illustrated in part E are used in a cell-based screen of a chemical compound library.

FIGS. 3A-3D depict characteristics and sequence information for particular phosphatases used to generate bindingzymes in embodiments described below. Each nucleotide sequence identified in FIGS. 3A-3D may be accessed at the http address http://www.ncbi.nlm.nih.gov/entrez/query.fcgi?db=Nucleotide. All nucleotide sequences referenced and accessed by the parameters set forth in FIGS. 3A-3D are incorporated herein by reference. Also incorporated herein by reference are amino acid sequences of polypeptides encoded by the referenced nucleotide sequences.

FIG. 4A shows information pertaining to bindingzyme counterparts of certain phosphatases described in FIGS. 3A-3D. For example, specific mutations in the corresponding native protein are shown for specific bindingzymes (see “mutant I” and “mutant 2” columns) and expected molecular weights corresponding to bindingzyme fusion proteins with glutathione S-transferase (GST) or maltose binding protein (MBP) are shown (see “MW fusion proteins” column). FIG. 4B shows polymerase chain reaction (PCR) primers useful for generating phosphatase coding regions and the mutagenesis oligodeoxyribonucleotides used to create the corresponding bindingzymes. All nucleotide sequences referenced and accessed by the parameters set forth in FIGS. 3A-3D with one or more mutations set forth in FIG. 4A are incorporated herein by reference. Also incorporated herein by reference are amino acid sequences of polypeptides encoded by such nucleotide sequences.

FIG. 5 illustrates specific embodiments described herein for constructing and using bindingzyme arrays.

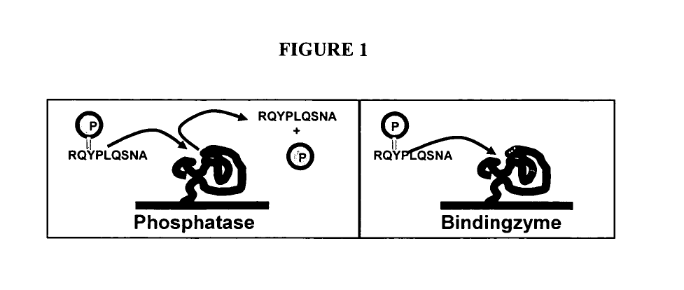

### DETAILED DESCRIPTION

In the methods, arrays, and systems described herein, a catalytically inactivated enzyme (i.e., a bindingzyme) is utilized as a substrate-specific binding protein. The bindingzyme is a modified enzyme that retains substrate binding but does not retain the ability to substantially modify or act on the substrate catalytically. In certain embodiments, the binding site in the substrate in contact with the bindingzyme includes a modification capable of being added by a native post-translational process, such as a phosphoryl modification for example.

These bindingzymes can bind to one or more polypeptide or peptide substrates in a biological sample and a signal corresponding to a bound substrate can be detected. Because only signals corresponding to the bound substrates are typically detected, these screens lead to relatively simple signal patterns and can be rapidly processed in a high throughput format. Also, the signal acts as a marker and samples can be distinguished from one another by detecting different signal levels corresponding to different levels of bindingzyme substrates in the samples without more information concerning a bindingzyme substrate. This feature also allows for high throughput processing as only signal information is required for informative screens. It is possible, however, to further characterize each substrate bound to a particular bindingzyme. For example, the amino acid sequence of a substrate from a sample bound to a bindingzyme may be deduced and the location of the post-translational modification in the substrate can be determined using routine methods (e.g. LC/MS/MS, which is described below).

Two or more samples may be contacted with an array of different bindingzymes, providing an advantage of rapidly determining differences in bindingzyme substrate levels between the samples. A panel of bindingzymes also can be used to rapidly screen a given set of biological samples. For example, to determine variances in the levels of one or more regulatory phosphoproteins in a group of biological samples, the group of samples can be rapidly screened across arrays of phosphatase-derived bindingzymes.

In initial screens, a bindingzyme in a bindingzyme array may detect substrate level variances in different biological samples, and such “informative” bindingzymes can be selected for subsequent screens. For example, informative bindingzymes identified in a screen of a particular group of biological samples can be selected, grouped in an array, and the array can be utilized in a high throughput screen for identifying test compounds that modulate levels of bindingzyme substrates in the particular group of samples or screen other biological samples for bindingzyme substrate variances. Non-informative bindingzymes (i.e. bindingzymes that do not detect substrate variances among samples because no signals or the same signals are detected when screened against different biological samples) also may be selected and grouped in an array to rapidly assess test compound specificity. The bindingzyme methods described herein are useful for isolating substrates for their identification, for detecting substrates in a sample, for discovering new ethical therapeutic drug candidates, and for assessing specificity and toxicity of therapeutic drug candidates, for example.

Bindingzymes

A bindingzyme is a modified enzyme that retains significant binding affinity for one or more substrates that normally bind to the non-modified enzyme with concomitantly reduced catalytic activity for the bound substrate. A bindingzyme binds to a substrate with more than or equal to 10-fold less affinity, sometimes more than or equal to 5-fold less affinity, and often more than or equal to 2-fold less affinity as compared to the non-modified enzyme. A bindingzyme also may bind to the substrate with the same or better affinity as compared to the non-modified enzyme. Substrate affinity can be quantified by comparing appropriate parameters such as Kₘ, K_{d}, on rates and/or off rates, for example. Catalysis typically is reduced 50-fold or more, often 100-fold or more, and sometimes 500-fold or more as compared to the non-modified enzyme. Catalytic rates can be quantified by comparing appropriate parameters such as a steady state maximum velocity or a pre-steady state kinetic constant, for example.

An enzyme substrate is a polypeptide, peptide, or other molecule that binds to the non-modified enzyme and is chemically altered by the enzyme. For example, a protease is an enzyme that binds to and hydrolyzes a bond in a peptide or polypeptide substrate. A protein phosphatase is an enzyme that binds to a phosphorylated polypeptide or peptide and removes one or more phosphoryl moieties. Protein phosphatases are characterized as protein tyrosine phosphatases, protein serine/threonine phosphatases, and dual specificity or multispecificity protein phosphatases according to which amino acid in the polypeptide or peptide they remove the phosphoryl moiety (i.e. tyrosine, serine/threonine, or either, respectively). A protein kinase adds a phosphoryl moiety to a polypeptide or peptide substrate. The bindingzyme may retain binding affinity for polypeptide substrates or peptide fragments thereof. Peptide fragments include the binding site described below and typically are 5 or more amino acids in length, often 10 or more, 15 or more, 20 or more, or 25 or more amino acids in length, and sometimes 30 or more, 40, or more, or 50 or more amino acids in length.

The binding site to which the bindingzyme binds in the substrate includes a substrate modification that is capable of being added to the substrate by a native post-translational modification process. Generally, native post-translational modification processes occur naturally in cells and involve enzymes that modify a polypeptide after it is synthesized (i.e. translated). Native post-translational modification processes include those capable of adding a phosphoryl, alkyl (e.g. methyl), fatty acid (e.g. myristoyl or palmitoyl), glycosyl (e.g. polysaccharide), an acetyl or peptidyl (e.g. ubiquitin) moiety to an enzyme substrate. Thus, a bindingzyme may be derived from an enzyme that removes such moieties, such as a protein phosphatase, a protein demethylase, an deacetylase, an enzyme that cleaves fatty acids from a protein substrate, a glycosylase, or an enzyme that removes ubiquitin from a protein, for example.

Any method that catalytically inactivates an enzyme, but retains the substrate binding activity of the enzyme can be used to produce a bindingzyme. Specific amino acid mutations that produce catalytically inactivated enzyme while retaining substrate-binding activity typically are utilized to generate bindingzymes. Amino acid substitutions can be introduced by site directed mutagenesis procedures and by mutation scanning techniques known in the art to identify appropriate mutations. Kits with explicit directions for making specific site-directed mutations are commercially available (e.g. Strategene (QuikChange® and QuikChangeXL®) and Clontech (BD Transformer™ Site-Directed Mutagenesis Kit)). DNA sequencing is routinely performed to verify that desired mutations have been introduced. Also, co-factors (if they exist) can be withheld and reaction conditions can be altered to produce bindingzymes, so long as the catalytic activity is abolished or greatly diminished while the binding activity is retained.

In a specific embodiment, phosphatase-derived bindingzymes are produced, arranged in an array, and utilized in high throughput screens of biological samples. Alkaline phosphatases proceed through a phosphoserine intermediate (e.g., J. H. Schwartz and F. Lipmann (1961) Proc. Natl. Acad. Sci. U.S.A. 47: 1996-2005) and some acid phosphatases form a phosphohistidine intermediate (e.g., R. L. VanEtten (1982) Ann. N.Y. Acad. Sci. 390: 27-51). In both types of enzymes, the critical serine and histidine amino acids that form the intermediate have been identified. For the class of protein phosphatase, it is a cysteine residue in the active site of protein tyrosine phosphatases (PTPs) that is critical for catalysis. It has been shown that mutation of the active site cysteine to serine in a PTP abolishes catalytic activity, but binding activity is retained (e.g., K. L. Guan and J. E. Dixon (1991) JBC 266: 17026-17030). The amino acid sequence HCXAGXXR is highly conserved among PTPs, and in specific embodiments mutation of the cysteine in this sequence (denoted “C”) to a serine is a strategy for generating bindingzymes derived from PTPs. While this active site cysteine can be modified to generate PTP-derived bindingzymes, other amino acids may be modified and resulting mutant enzymes can be routinely screened for meeting bindingzyme criteria of substrate binding and impaired catalysis (e.g., A. J. Flint, T. Tiganis, D. Barford, and N. K. Tonks, Proc. Natl. Acad. Sci. USA, Vol. 94:1680-1685, March 1997; L. Xie, Y L Zbang, and Z Y Zhang, Biochemistry, Vol 41: 4032-4039). Similarly, other amino acids in protein serine/threonine phosphatases and dual specificity or multispecificity protein phosphatases can be mutated and the resulting mutant enzymes can be routinely screened for bindingzyme characteristics.

Depending upon the bindingzyme generated, an expression systems is utilized, such as in bacteria, yeast, baculovirus, or mammalian systems, for example. Bacterial expression is preferred when possible because of the ease of use and high levels of expression. Bindingzymes can be produced as fusion proteins to facilitate their capture for purification and use in assays, where they can be captured by binding of the fusion protein to one or its substrates. Examples of fusion moieties are maltose binding protein (MBP), glutathione-S-transferase (GST); and polyhistidine (His) linked to the bindingzyme. All are commercially available and with commercially available supporting reagents. The plasmid pMAL-c2X (New England BioLabs) can be used to generate MBP fusions; the plasmids pGEX-2T and pGEX-6P-1 (Amersham Biosciences) can be used to generate GST fusions; and the vector pHAT (Clontech) can be used to generate His fusions. For all three, affinity purification reagents are available (Pierce), as are coated microwell plates for linking the bindingzymes to solid supports, which is further described hereafter.

Bindingzyme Systems and Arrays

A system can be any solid support arrangement adapted to contain a liquid medium. As used herein, the term “system” refers to an environment that receives the assay components, which includes, for example, microtiter plates (e.g., 96-well or 384-well plates), silicon chips having molecules immobilized thereon and optionally oriented in an array (see, e.g., U.S. Pat. No. 6,261,776 and Fodor, Nature 364: 555-556 (1993)), microfluidic devices (see, e.g., U.S. Pat. Nos. 6,440,722; 6,429,025; 6,379,974; and 6,316,781), and cell culture vessels such as Petri dishes and eight-well plates. The system can include attendant equipment for carrying out the assays, such as signal detectors, robotic platforms that move solid supports from one location to another, and pipette dispensers.

One embodiment of a system is an array of bindingzymes, typically oriented in two dimensions on a solid support. Arrays of bindingzymes sometimes are referred to herein as a bindingzyme “panel.” In an embodiment, the array is oriented in a microtiter plate where each well contains the same bindingzyme compared to another well or a different bindingzyme compared to another well. As used herein, the term “a bindingzyme” refers to one or more bindingzymes, and accordingly, a well in a microtiter plate array, for example, sometimes contains one bindingzyme and sometimes contains two or more bindingzymes. Where an array (e.g., one or more wells in a microtiter plate) includes two or more bindingzymes, the array sometimes includes 2 or more, 3 or more, 4 or more, 5 or more, 6 or more, 7 or more, 8 or more, 9 or more, 10 or more, 11 or more, 12 or more, 13 or more, 14 or more or 15 or more bindingzymes. In certain embodiments, only informative bindingzymes are arranged in an array, and in other embodiments, the array includes a mixture of informative and non-informative bindingzymes, or only non-informative bindingzymes. For example, a well in a microtiter plate array sometimes includes one or more informative bindingzymes; one or more non-informative bindingzymes; one non-informative bindingzyme; one informative bindingzyme; two or more non-informative bindingzymes; two or more informative bindingzymes; and sometimes includes a mixture of one or more informative bindingzymes and one or more non-informative bindingzymes. Informative and non-informative bindingzymes are described herein. In microtiter plate embodiments in which one or more wells include one or more informative bindingzymes, all of the informative bindingzymes identified may be included in a well or may be distributed among several wells, such as 2, 3, 4 or 5 wells for example (e.g., where 10 informative bindingzymes are identified, each of five wells in a microtiter plate may include two different informative bindingzymes). The bindingzymes may be distributed among different wells in a random manner, a redundant manner (see below), and may be distributed according to the magnitude of signals generated by the bindingzymes (e.g., bindingzymes giving rise to signals of high amplitude may be grouped together in a well of a microtiter plate), for example.

In specific embodiments, the panel includes bindingzymes derived from one or more protein tyrosine phosphatases, protein serine/threonine phosphatases, and dual specificity protein phosphatases, or combinations thereof. Such panels offer an advantage of rapidly screening biological samples across multiple protein phosphatase-based bindingzymes.

In certain embodiments, the array includes bindingzyme redundancy. In a microtiter plate array, for example, a well may include a first bindingzyme and a second bindingzyme, a second well may include the same second bindingzyme and a third bindingzyme, and a third well may include the same third bindingzyme and a fourth bindingzyme. The wells also may include other bindingzymes. The signals generated by each well can be compared (e.g., mass spectrometry signals corresponding to components bound by the bindingzymes). In arrays containing bindingzyme redundancy, signals corresponding to redundant bindingzymes are useful for determining relative signal intensities, determining relative signal areas, determining relative signal positions, determining relative signal shapes, and normalizing differences between signals.

In certain embodiments, bindingzyme arrays include internal standards. In an embodiment, an array includes one or more bindingzymes that generate a predetermined range of signal intensities and one or more bindingzymes giving rise to unknown signal intensities. In such an embodiment, the bindingzymes used as internal standards sometimes are informative (e.g., one or some of the signals in a pattern for an informative bindingzyme sometimes do not change) and often are non-informative. Where two or more bindingzymes are utilized as internal standards, the bindingzymes often are selected to give rise to a range of signal intensities. For example, a bindingzyme array may include three standard bindingzymes that yield high, medium and low signal intensities, respectively. Other arrays may include four or more internal standard bindingzymes to yield finer gradations in the signal intensity range. In microtiter plate array embodiments, a well sometimes includes one or more bindingzymes giving rise to predetermined signal intensities and one or more bindingzymes giving rise to unknown signal intensities.

Non-informative bindingzymes in arrays that include informative bindingzymes are useful as internal standards as described above and in other applications. In certain embodiments, non-informative bindingzymes in an array can be utilized to determine selectivity of a test molecule identified by informative bindingzymes. In such embodiments, a test molecule is identified as being selective where the majority of uninformative bindingzymes remain uninformative when a biological sample is contacted with the test molecule. In certain embodiments, 99% or more, 95% or more, 90% or more, 85% or more, 80% or more, 75% or more, 70% or more, 60% or more or 50% or more of the uninformative bindingzymes remain uninformative when a biological sample is contacted with the test molecule. These embodiments for determining test molecule selectivity are applicable, for example, to embodiments for identifying molecules that effect similar responses as reference compounds (e.g., EPO, TNF-alpha, a reference antineoplastic compound or a reference antimetastatic compound) and reducing toxicity of certain compounds (e.g., a hepatotoxin), which are described hereafter.

A bindingzyme may or may not be immobilized to a solid support before it is contacted with a biological sample and/or test molecule. A free bindingzyme may be separated from other assay components after it is contacted with a biological sample or test molecule, for example, by virtue of a cleavable or non-cleavable tag that has affinity for a chemical moiety on a solid support or that can be chemically linked to a solid support. Alternatively, a free bindingzyme may be lipophilic and it can be separated into a lipophilic environment after it is contacted with a biological sample or test molecule.

A bindingzyme can be immobilized to a solid support by any convenient technique known in the art. The attachment between the bindingzyme and the solid support may be covalent or non-covalent (see e.g. U.S. Pat. No. 6,022,688 for non-covalent attachments). The solid support may be one or more surfaces of the system, such as one or more surfaces in each well of a microtiter plate, a surface of a silicon wafer, a surface of a bead (see e.g. Lam, Nature 354: 82-84 (1991)) that is optionally linked to another solid support, or a channel in a microfluidic device, for example. Examples of linking agents (e.g. photocleavable linkers and chemically cleavable linkers), functional groups, suitable solid supports, and tools for applying polypeptides to a solid support are described in U.S. Pat. No. 6,387,628 (Little et al.). Solid supports, linker molecules for covalent and non-covalent attachments, and methods for immobilizing molecules to solid supports also are described in U.S. Pat. Nos. 6,261,776; 5,900,481; 6,133,436; and 6,022,688; and WIPO publication WO 01/18234.

The bindingzyme system or array can be contacted with a biological sample or a test molecule in any convenient manner. Contacting these assay components with one another can be accomplished by adding the biological sample and/or test molecule to the same reaction vessel containing the bindingzyme, for example, and the components in the system may be mixed in variety of manners, such as by oscillating a vessel, subjecting a vessel to a vortex generating apparatus, repeated mixing with a pipette or pipettes, or by passing fluid containing one assay component over a surface having another assay component immobilized thereon, for example. In an embodiment, a bindingzyme in one system may be contacted with one biological sample and/or test compound and the same bindingzyme in another system may be contacted with another biological sample and/or test compound (e.g. a bindingzyme in one well of a microtiter plate may be contacted with one biological sample and another well containing the same bindingzyme in another microtiter plate may be contacted with another biological sample). The bindingzymes in the system or array can be contacted by the biological samples and/or test molecules in any order and for any amount of time. The bindingzyme and the biological sample and/or test molecule may be contacted with one another for 1 minute or less, 15 minutes or less, 30 minutes or less, 30 minutes or less, one hour or less, 6 hours or less, 12 hours or less, 24 hours or less, or 48 hours or less.

After the bindingzymes are contacted with the biological sample and/or test compound, the mixture may be subjected to further processing. For example, the mixture may be subjected to washing steps for removing components in the biological sample not substantially bound to the bindingzyme from the system. Bindingzyme/substrate complexes also may be contacted with agents that modify the bindingzyme or substrate (e.g. one or more proteases (e.g. exoproteases and/or endoproteases), one or more protease inhibitors, or agents that cleave the linkage between the bindingzyme and the solid support to which it is immobilized). Substrate(s) bound to bindingzyme also may be eluted and separated into another system amenable for signal detection and analysis. For example, the bound substrates may be eluted by contacting the bindingzyme/substrate complex with a solution comprising a high concentration of salt (e.g. 1 M NH₄Cl or a more concentrated NH₄Cl solution), the substrate elutes from the immobilized bindingzyme into the salt solution, and the eluted substrate can be deposited onto a solid support having matrix for MALDI-TOF signal analysis directly or after further processing steps (e.g. an eluate may be subjected to a sample conditioning step before being deposited on a solid support for MALDI-TOF analysis). A substrate bound to a bindingzyme also may be eluted and subjected to amino acid sequencing procedures, as described below, which can deduce a full or partial amino acid sequence for the bindingzyme substrate, determine to which amino acid a post-translational modification moiety is attached, and characterize the modification moiety. A substrate also may be contacted with an agent that removes a moiety capable of being added by a native post-translational process from the substrate when the substrate is bound to the bindingzyme or eluted from the bindingzyme (e.g. the substrate may be treated with a phosphatase that removes one or more phosphoryl moieties prior to signal detection), for example.

Bindingzyme arrays can be utilized to screen biological samples and test molecules in a high throughput manner. For example, an array comprising informative bindingzymes can be utilized to screen test molecules at a rate of 100 or more test molecules per day; 500 or more test molecules per day, 1,000 or more test molecules per day; 5,000 or more test molecules per day or 10,000 or more test molecules per day.

Biological Samples

The assay methods described herein can be utilized to detect a polypeptide variance between two or more different biological samples. Biological samples often are derived from organisms, tissues, or cells, and examples include whole cells, disrupted cells (e.g. cell lysates), and purified cell fractions (e.g. a purified polypeptide). Biological samples sometimes are synthetically manufactured, such as an in vitro translated polypeptide, an isolated recombinant polypeptide, or an isolated chemically synthesized peptide, for example. Biological samples, often synthetically manufactured polypeptides or peptides, sometimes are treated with agents that add or subtract a post-translational modification (e.g. a phosphopolypeptide or phosphopeptide may be treated with a protein phosphatase that removes a phosphoryl modification) and the biological sample may be treated with such agents before, during, or after they are contacted with a bindingzyme and/or a test molecule.

Differences among cell-based biological samples may be naturally occurring (e.g. metastatic cells or neoplastic cells may be compared to non-metastatic or neoplastic counterparts) and differences in cells may be induced (e.g. a recombinant polypeptide is expressed in a cell, a cell is cultured with or without a growth medium, or a cell is treated with an agent (e.g. a differentiation-inducing or proliferation-inducing agent (e.g. erythropoietin (EPO)) or a toxin (e.g. hepatic toxin)). Biological samples also may be treated with such agents as proteases (e.g. exoproteases and/or endoproteases) or with one or more protease inhibitor agents known in the art, for example. In certain embodiments, the biological samples are not treated with wide-spectrum inhibitors, such as a general phosphatase inhibitor (e.g., pervanadate).

Detecting and Comparing Signals

As noted above, a signal corresponding to a substrate bound to a bindingzyme typically is detected (e.g. unbound substrate is washed away from the bound substrate), which advantageously leads to relatively clean signal patterns. These clean signal patterns allow for less ambiguous signal comparisons and reduce the amount of time required for signal analysis, which allows for the assays to be carried out in a high throughput manner.

Any signal generating molecule and signal detection technique known in the art can be used to detect and compare signals corresponding to one or more polypeptide bound to the bindingzyme. For example, a fluorescent signal may be monitored in the assays by exciting a fluorophore at a specific excitation wavelength and then detecting fluorescence emitted by the fluorophore at a different emission wavelength. Many fluorophores and their attendant excitation and emission wavelengths are known in the art (e.g. Anantha et al., Biochemistry 37: 2709-2714 (1998); Qu & Chaires, Methods Enzymol 321:353-69 (2000)). Standard methods for detecting fluorescent signals are also known in the art, such as by using a commercially available fluorescence detector. Background fluorescence may be reduced in the system with the addition of photon reducing agents (see e.g. U.S. Pat. No. 6,221,612), which can enhance the signal to noise ratio. Assays may employ other types of signal molecules, such as a radioactive isotope (e.g., ¹²⁵I, ¹³¹I, ³⁵S, ³²P, ¹⁴C or ³H); a light scattering label (Genicon Sciences Corporation, San Diego, Calif. and see e.g. U.S. Pat. No. 6,214,560); an enzymic or protein label (e.g. GFP or peroxidase); a chromogenic label or dye (e.g. Texas Red); or a stain (e.g. silver-staining polypeptides in a 1D or 2D polyacrylamide electrophoresis gel or capillary electrophoresis gels). Also, other signals may be detected, such as NMR spectral shifts (see e.g. Arthanari & Bolton, Anti-Cancer Drug Design 14: 317-326 (1999)), fluorescence resonance energy transfer (see e.g. Simonsson & Sjöback, J. Biol. Chem. 274: 17379-17383 (1999)), or circular dichroism signals.

Another signal that can be detected is a change in refractive index at a solid optical surface, where the change is caused by the binding or release of a refractive index enhancing molecule near or at the optical surface. These methods for determining refractive index changes of an optical surface are based upon surface plasmon resonance (SPR). SPR is observed as a dip in light intensity reflected at a specific angle from the interface between an optically transparent material (e.g., glass) and a thin metal film (e.g., silver or gold). SPR depends upon the refractive index of the medium (e.g., a sample solution) close to the metal surface. A change of refractive index at the metal surface, such as by the adsorption or binding of material near the surface, will cause a corresponding shift in the angle at which SPR occurs. SPR signals and uses thereof are further exemplified in U.S. Pat. Nos. 5,641,640; 5,955,729; 6,127,183; 6,143,574; and 6,207,381, and WIPO publication WO 90/05295 and apparatuses for measuring SPR signals are commercially available (Biacore, Inc., Piscataway, N.J.). In one embodiment, a bindingzyme can be linked via a linker to a chip having an optically transparent material and a thin metal film, and interactions between the immobilized bindingzyme and test compounds and/or biological samples added to the system can be detected by changes in refractive index.

A signal often detected in the methods described herein is a mass spectrometric signal. Examples of mass spectrophotometric techniques are listed in U.S. Pat. No. 6,387,628, supra, including ionization techniques such as matrix assisted laser desorption (MALDI), continuous or pulsed electrospray (ESI) and related methods such as ionspray or thermospray, and massive cluster impact (MCI), and detection techniques such as linear or non-linear reflectron time-of-flight (TOF), single or multiple quadrupole, single or multiple magnetic sector, Fourier transform ion cyclotron resonance (FTICR), ion trap, and combinations thereof. Mass spectrometric signals can be measured using a commercially available mass spectrophotometer (e.g. Bruker Daltonics manufactures MALDI-TOF instruments) and the mass spectrophotometer can be combined with an array described herein in a system.

In an embodiment, the mass spectrometric signal is a MALDI-TOF signal, which often is utilized for separating signals corresponding to components of a biological sample bound to a bindingzyme. MALDI-TOF signals and methods of detecting them are well-characterized in the art and methods for enhancing signal intensity and resolution (e.g. conditioning methods) also are known (see e.g. U.S. Pat. No. 6,387,628, supra). MALDI-TOF signals for informative bindingzymes can differ in at least two respects. A signal corresponding to a substrate having the same mass may have a different amplitude for different biological samples, which is indicative of different levels of a substrate in each sample. Also, a signal corresponding to a substrate having a different mass may be present in one sample and not another, which is indicative of a different substrate present in one biological sample and not in another. One substrate bound to a bindingzyme often yields one signal, and therefore, a signal pattern may be detected for one bindingzyme because more than one substrate in a sample may bind to each bindingzyme.

In another embodiment, the mass spectrometric signal is a LC/MS/MS signal, which often is utilized for determining the amino acid sequence of a peptide bound to a bindingzyme as well as which amino acid or amino acids in the peptide are phosphorylated. Methods and instruments for carrying out LC/MS/MS are known in the art (see e.g. U.S. Pat. Nos. 4,982,097 and 6,027,890, and H. Zhou, J. D. Watts, and R. Aebersold, Nature Biotechnology, Vol 19: 375-378, April 2001).

After signals in each system are detected, the signals are compared to one another. The signals may be compared by eye and comparisons may be facilitated by commercially available software typically manufactured for use with the equipment that detects the signal (e.g. software for comparing MALDI-TOF spectrometric data is commercially available from Bruker Daltonics). Commercially available software also may be modified for specific comparisons and new software may be developed. As noted above, a signal pattern comprising more than one signal may be detected for a bindingzyme, and the individual signals may be compared to corresponding signals for another system (e.g. a well in a microtiter plate containing another bindingzyme, biological sample, or test molecule) and/or the pattern itself may be compared.

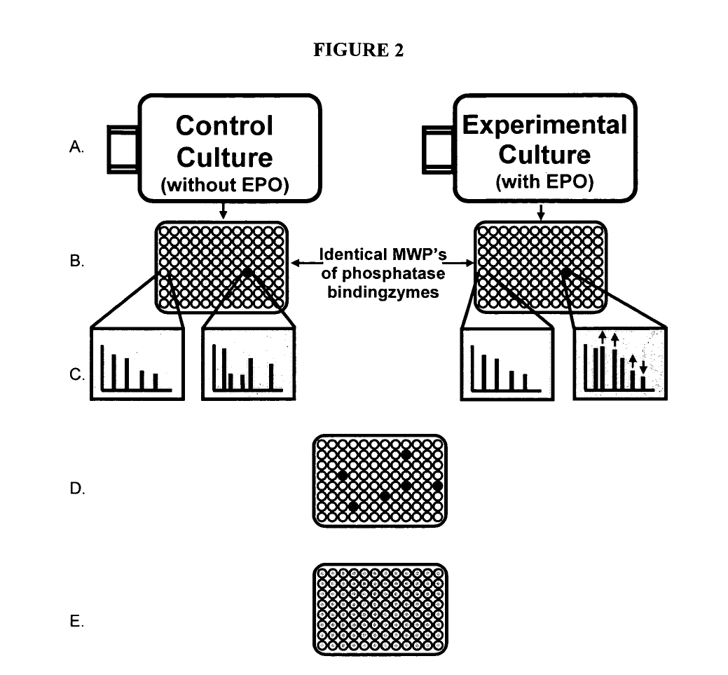

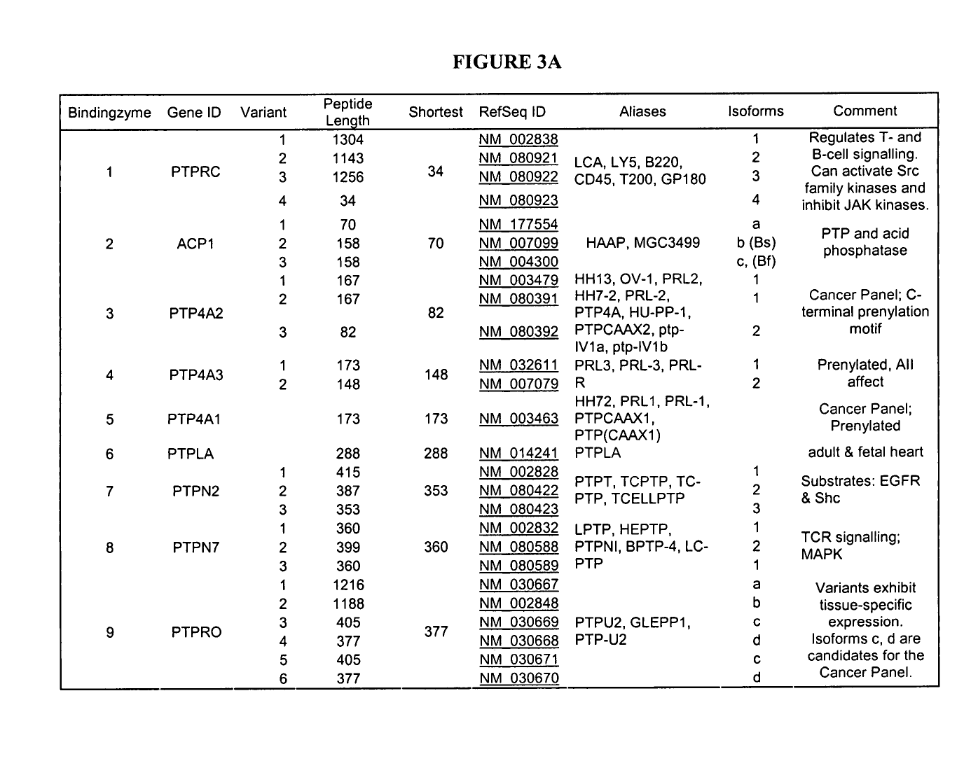

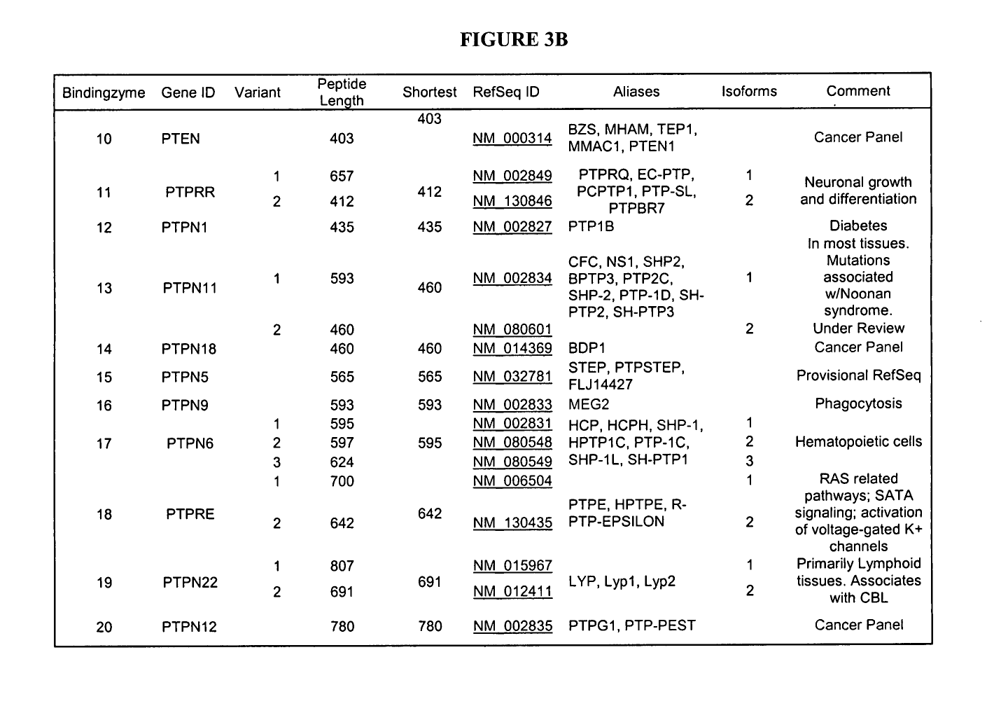

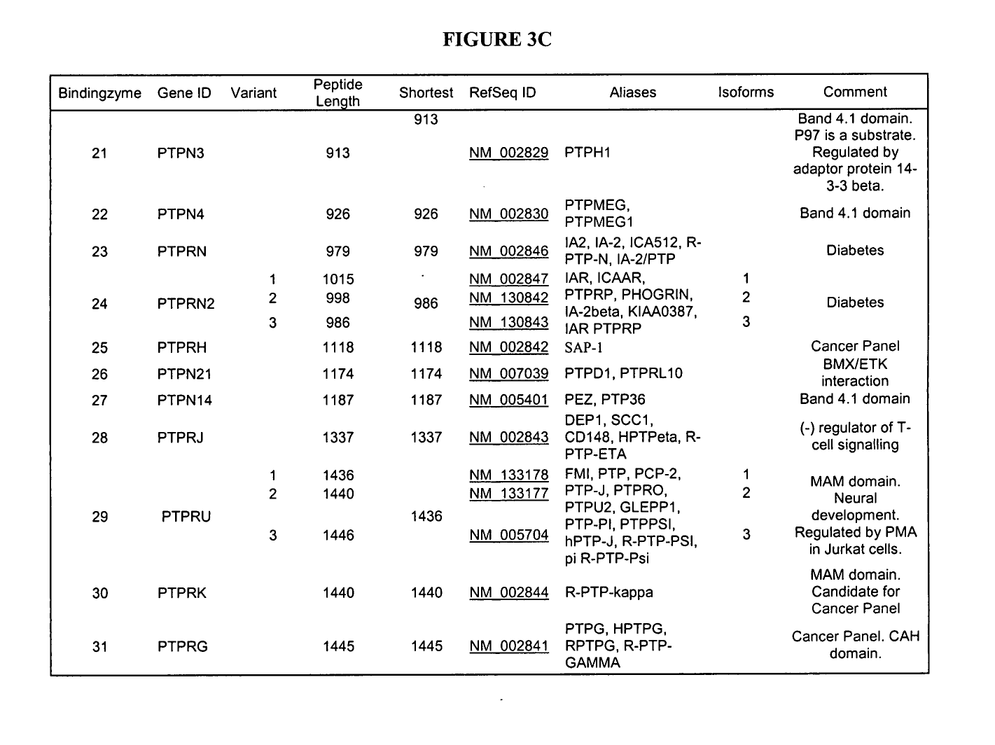

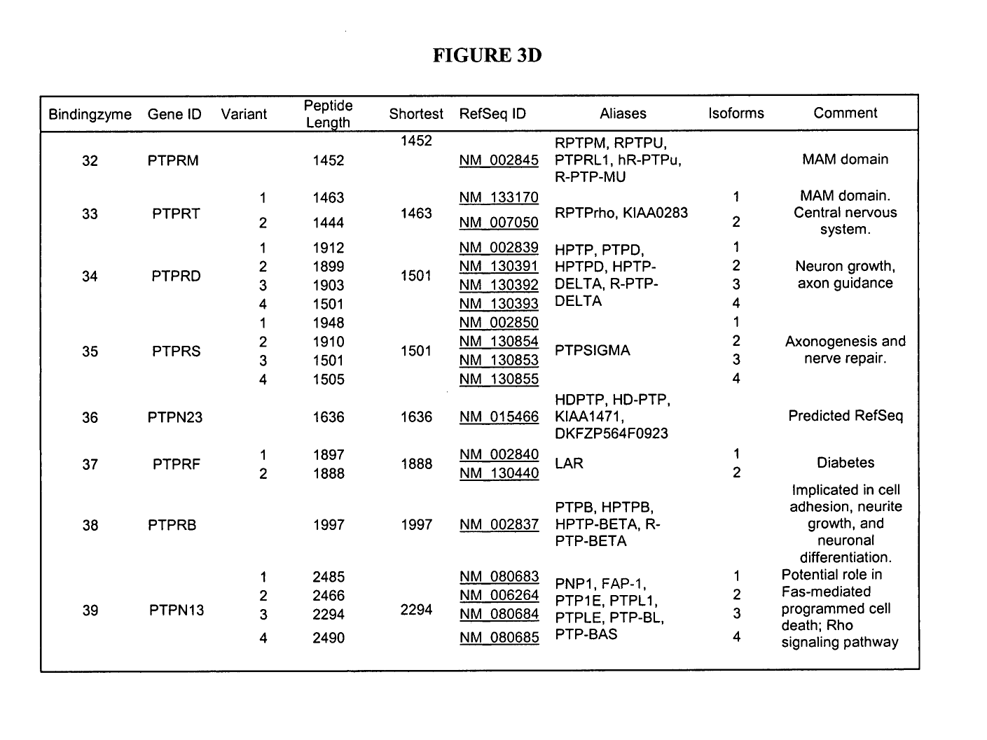

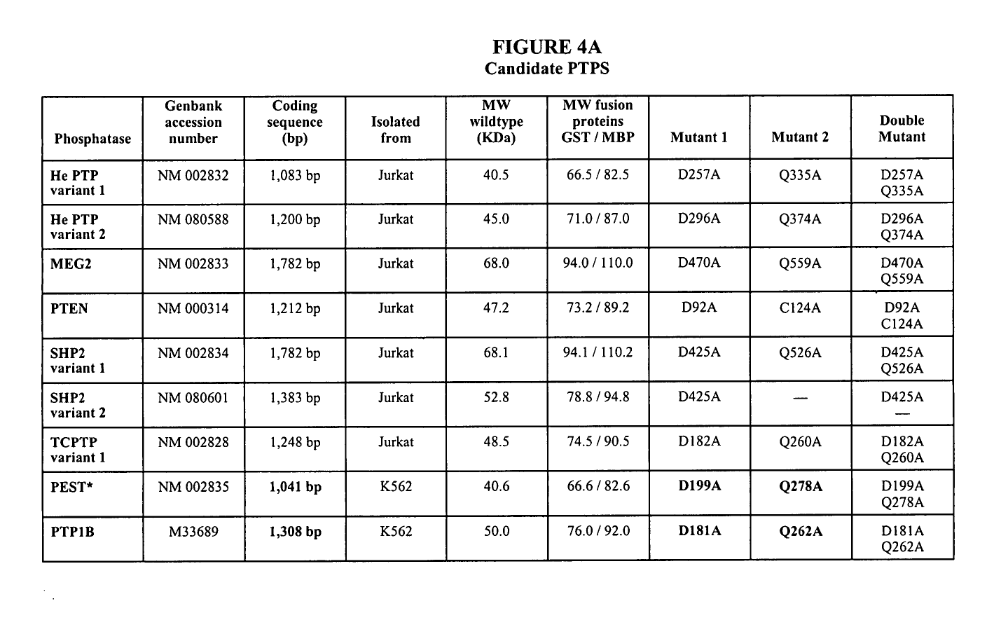

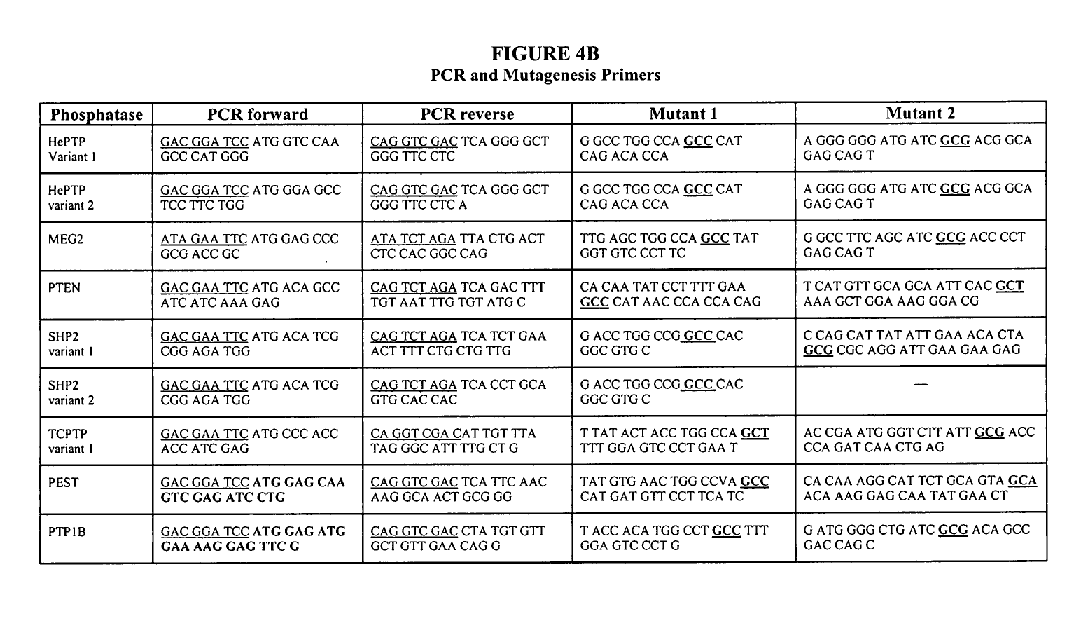

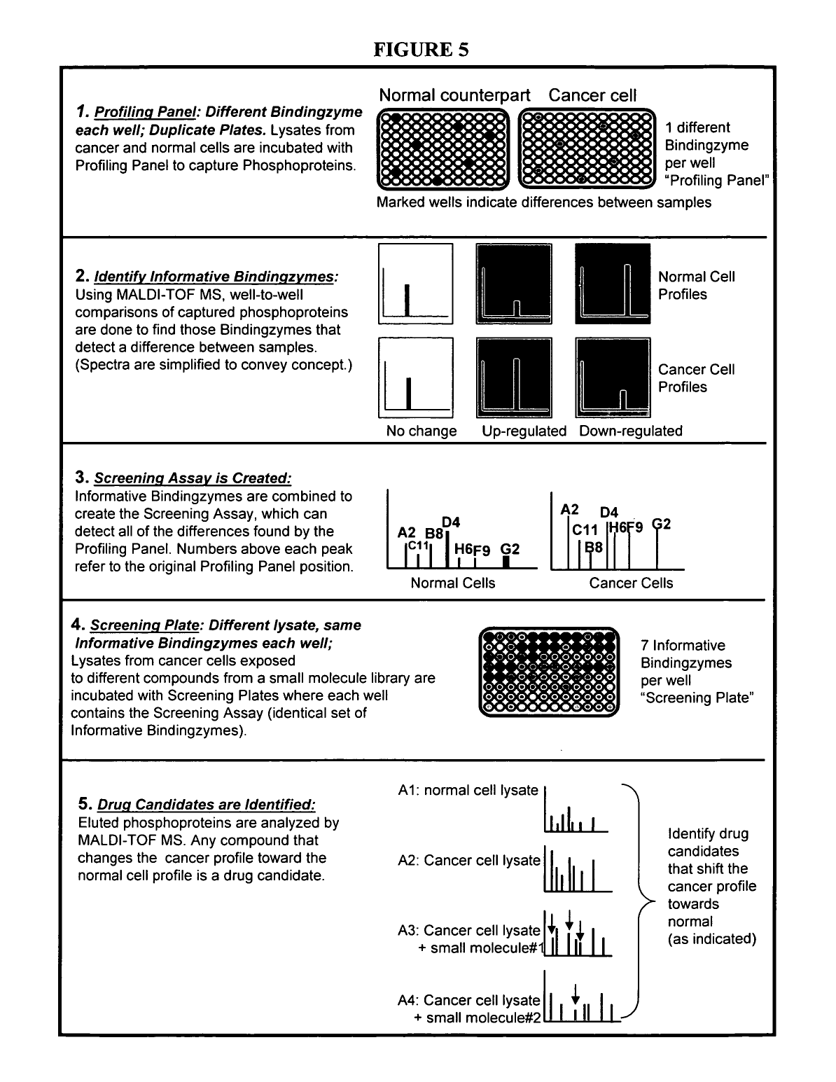

FIG. 2 illustrates an embodiment in which signals derived from two samples screened across a bindingzyme array are compared. For one bindingzyme, the MALDI-TOF signals and signal pattern do not vary (non-darkened wells) with respect to signal amplitude (height) and signal location (mass). Such a signal comparison demonstrates that the particular bindingzyme is non-informative for the biological samples screened. It is noted, however, that the signal variations may exist for the same bindingzyme when different biological samples are screened, and therefore, that same bindingzyme may be informative in other screens. For another bindingzyme in FIG. 2, part B (darkened), signal amplitudes vary for certain masses detected. This signal variance often is a result of a different level of a phosphopeptide present in one sample as compared to another sample. The darkened bindingzyme is informative for the particular group of biological samples in FIG. 2, part B, as it detects a signal variance. A variance in a signal comparison is detected when the amplitude, position, or shape of a signal in one system differs from another in a corresponding system sometimes by 15% or more or 20% or more and often by 25% or more, 30% or more, 50% or more, or 75% or more. A signal pattern from a bindingzyme may be considered informative when the amplitude, position, or shape of one signal among the other signals in the pattern differs from a corresponding signal or lack of a signal in a comparative pattern. Also, a signal pattern from a bindingzyme sometimes is considered a substantially dissimilar match to another signal pattern when only one signal among two or more other signals differs.

Bindingzyme arrays and the screening methods described above can be utilized for conducting diagnostic assays. For example, biological samples corresponding to a diseased state and non-diseased state (e.g. metastatic cells and non-metastatic counterparts) can be screened across an array of protein phosphatase-derived bindingzymes and informative bindingzymes can be selected for the particular screen. The same array of bindingzymes or a newly constructed array of informative bindingzymes then can be screened with blood or tissue samples from patients to determine if those samples exhibit a signal pattern that matches the signal pattern for the initially screened diseased samples or non-diseased samples.

Informative and uninformative bindingzymes can be selected and arranged in an array for further screens carried out in a similar manner as described above. Such arrays can be utilized in a high throughput format to identify and optimize new therapeutic molecules as described below.

Methods For Identifying and Optimizing Lead Therapeutic Modulators

Molecules that modulate enzyme/substrate interactions and levels of bindingzyme substrates in a biological sample can be identified and optimized by screening test molecules using the assays described herein. Test molecules can be added to the array in any order with respect to the biological sample and the test molecule may be added to the biological sample before the latter is added to the array. Test molecules often are identified as potential therapeutic molecules when the test molecule induces a signal, two or more signals, and/or a signal pattern that matches or is substantially similar to those corresponding to a non-disorder biological sample or a biological sample that has undergone a therapy. For example, a potential therapeutic molecule often is one that modulates a polypeptide variance between two biological samples, and a test molecule that modulates the difference between the signals relative to the difference in the absence of the test compound often is identified as a molecule that modulates a polypeptide variance between two biological samples. A molecule that modulates a polypeptide variance sometimes reduces the difference between the signal from the first system and the signal from the second system and sometimes negates the difference between the signals in the two systems (e.g., there is no detectable difference between the corresponding signals).

For example, signals can be compared between a first biological sample contacted with a reference compound such as EPO and a second biological sample contacted with a test molecule using an informative bindingzyme array developed from initial screens of biological samples administered EPO or not administered EPO. The informative bindingzymes selected for the array are those that detect a signal variance in the presence or absence or EPO. Test molecules that elicit signals that match or are substantially similar to the signal pattern elicited by EPO administration are identified as potential therapeutic alternatives to EPO. An embodiment for identifying EPO alternatives is described in Example 1. Similarly, an embodiment for identifying TNF-alpha antagonists is described in Example 2.

Similar screens can be carried out for identifying antineoplastic and antimetastatic molecules. Such screens are processed using informative bindingzyme arrays developed from an initial screen in which metastatic or neoplastic cell samples are compared with non-cancerous counterpart cells. The biological samples comprising cancerous cells sometimes include cells from a cell line derived from a cancerous tissue or directly from a cancerous tissue, for example. Test molecules screened using informative bindingzyme arrays are identified as antimetastatic modulators and antineoplastic modulators when the signals elicited by such molecules match or are substantially similar to those from non-cancerous cell samples. Example 3 describes an embodiment of this approach. Cell-based screens can be carried out using biological samples derived from a proliferating cell line or a subject having a cell proliferative disorder. Cell proliferative disorders include, for example, hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol/Hemotol. 11:267-97 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

The specificity of a potential therapeutic molecule and derivatives thereof can be assessed by using non-informative bindingzyme array screens as described above. Variances induced by the molecule or derivatives thereof are detected using arrays of non-informative bindingzymes selected in a previous screen. For example, non-informative protein phosphatase-derived bindingzymes identified in a +/−EPO screen (see e.g. Example 1) can be arranged in an array and screened in the presence of a potential therapeutic and/or derivatives thereof. Potential therapeutic molecules or derivatives that do not modify a signal or a signal profile, or modify the fewest signals compared to other molecules, are considered specific or most specific, respectively, for the particular array. Specificity assessments determined by non-informative bindingzyme array screens can be used in conjunction with other in vitro or in vivo data for determining specificity for a particular potential therapeutic or derivative. An embodiment of a screen for assessing specificity is described in Example 4.

Toxicity often is a concern when determining the therapeutically effective dose of a potential therapeutic or derivative. Bindingzymes identified as informative in screens of cell-based samples treated or not treated with one or more toxins (e.g., one or more hepatotoxins) can be selected and arranged in an array used to screen potential therapeutics or derivatives thereof. Examples of hapatotoxins are known in the art, and include iproniazid (MARSILID), ticrynafen (SELACRYN), benoxaprofen (ORAFLEX), bromfenac (DURACT), and troglitazone (REZULIN). Molecules that elicit signals or patterns that match or are substantially similar to those detected for cell samples not treated with a hepatotoxin are identified as less toxic as compared to others that elicit signals or patters similar to those detected for cell samples treated with a hepatotoxin. In these assays varying amounts of a molecule can be added to the biological sample or system to determine threshold concentrations that are toxic. A specific embodiment of a toxicity assessment screen is described in Example 5.

Other bindingzyme screens described herein also can be used to assess a therapeutically effective dose of a potential therapeutic. For example, the assay involving an array of informative bindingzymes described in Example 1 can be used to screen varying doses of a potentially therapeutic molecule to determine the minimum dose required to elicit a signal pattern that matches or is substantially similar to those of the +EPO (i.e., exposed to EPO) samples. In such embodiments, the potentially therapeutic molecule can be added to the biological sample or a subject from which the biological sample is isolated prior to contacting the biological sample with a bindingzyme.

Data obtained from in vitro cell culture assays and in vivo animal studies can be used in conjunction with bindingzyme assessments of toxicity when formulating a range of dosages for use in human subjects. The dosage of test molecules lies within a range of circulating concentrations that include an ED₅₀ with little or no toxicity. The dosage can vary within this range depending upon the dosage form employed and the route of administration utilized. The therapeutically effective dose of a test molecule can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in the subjects. Levels in plasma can be measured, for example, by high performance liquid chromatography. An effective dose of a test molecule can generally range from about 1.0 μg to about 5000 μg of peptide for a 70 kg subject. Toxicity and therapeutic efficacy of modulators can be determined by standard pharmaceutical procedures in cell cultures or experimental animals, for example, for determining an LD₅₀ value (the dose lethal to 50% of the population) and an ED₅₀ value (the dose therapeutically effective in 50% of the population). The dose ratio between toxic and therapeutic effects is the therapeutic index and it can be expressed as the ratio LD₅₀/ED₅₀. Test molecules that exhibit large therapeutic indices are preferred. While test molecules that exhibit toxic side effects can be used, care can be taken to design a delivery system that targets such molecules to the site of affected tissue in order to minimize potential damage to uninfected cells, thereby reducing side effects.

The assays described herein can be used to screen the effect of molecules on a purified polypeptide or peptide bindingzyme substrate. The sequence of a bindingzyme substrate may be determined by methods described herein, and the substrate may be synthesized. The substrate may be introduced to a bindingzyme array with or without a post-translational modification (e.g. substrate may include a phosphoryl moiety or not include the phosphoryl moiety). The substrate may be contacted with a bindingzyme array and can be used to screen molecules that inhibit or enhance interactions between the bindingzyme and the substrate, where modulators are identified as molecules that elicit signal variances. Specific enzyme-based screening embodiments are described in Examples 6 and 7. The substrate also can be contacted with an enzyme that acts upon it, such as a protein kinase or a protein phosphatase, and the molecules can be added to determine which of them modulate the interaction between the enzyme and the substrate. In the latter screens, interactions between the enzyme and the substrate can be monitored by detecting processed polypeptide or peptide (e.g. phosphorylated or dephosphorylated peptide or polypeptide) or by detecting the chemical moiety added to or removed from the substrate by the enzyme (e.g. released phosphate or added phosphate).

Thus, in certain embodiments, the sequence of a polypeptide bound to an inactivated enzyme is determined, sometimes by mass spectrometry (e.g., LC/MS/MS). After the sequence is determined, the corresponding polypeptide or a fragment of the polypeptide having the modification moiety sometimes is synthesized (e.g., with or without the modification moiety). In certain embodiments, the polypeptide having the modification moiety is contacted with an enzyme that removes the modification moiety, and the amount of modification moiety removed sometimes is detected. In some embodiments, the enzyme that removes the modification moiety is contacted with a test molecule and it is determined whether the test molecule modulates the removal of the modification moiety. In certain embodiments, the modification is a phosphoryl moiety and the polypeptide is contacted with a protein phosphatase. In other embodiments, a polypeptide without the modification moiety is contacted with an enzyme capable of adding to the polypeptide the modification moiety, and in some embodiments, polypeptide having the modification moiety is detected. In certain embodiments, the enzyme is contacted with a test molecule and it is determined whether the test molecule modulates the addition of the modification moiety to the polypeptide. In specific embodiments, the modification moiety is a phosphoryl moiety and the polypeptide is contacted with a protein kinase.

Test molecules often are organic or inorganic compounds having a molecular weight of 10,000 grams per mole or less, and sometimes having a molecular weight of 5,000 grams per mole or less, 1,000 grams per mole or less, or 500 grams per mole or less. Also included are salts, esters, and other pharmaceutically acceptable forms of the compounds. Compounds can be obtained using any of the combinatorial library methods known in the art, including spatially addressable parallel solid phase or solution phase libraries; synthetic library methods requiring deconvolution; “one-bead one-compound” library methods; and synthetic library methods using affinity chromatography selection. Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and Gallop et al., J. Med. Chem. 37: 1233 (1994).

A test molecule sometimes is a nucleic acid, a catalytic nucleic acid (e.g. a ribozyme), a nucleotide, a nucleotide analog, a polypeptide, an antibody, or a peptide mimetic. Methods for making and using such test molecules are known. For example, methods for making ribozymes and assessing ribozyme activity are described (see e.g. U.S. Pat. Nos. 5,093,246; 4,987,071; and 5,116,742; Haselhoff & Gerlach, Nature 334: 585-591 (1988) and Bartel & Szostak, Science 261: 1411-1418 (1993)). Also, peptide mimetic libraries are described (see e.g. Zuckermann et al., J. Med. Chem. 37: 2678-85 (1994)) and methods for generating antibodies are described (see e.g., Harlow & Lane, Antibodies, Cold Spring Harbor Laboratory Press, New York (1988)).

The test molecule may be formulated for a delivery to a subject from which the biological sample is derived and may be formulated for delivery to cells in the biological sample. The formulation can include pharmaceutically acceptable salts, esters, or salts of such esters of the test molecule. Formulations can be prepared as a solution, emulsion, or polymatrix-containing (e.g., liposome and microsphere). Examples of such compositions are set forth in U.S. Pat. No. 6,455,308 (Freier), U.S. Pat. No. 6,455,307 (McKay et al.), U.S. Pat. No. 6,451,602 (Popoff et al.), and U.S. Pat. No. 6,451,538 (Cowsert), and examples of liposomes also are described in U.S. Pat. No. 5,703,055 (Felgner et al.) and Gregoriadis, Liposome Technology vols. I to III (2nd ed. 1993). The formulation can be prepared for any mode of administration, including topical, oral, pulmonary, parenteral, intrathecal, and intranutrical administration. The formulations may include one or more pharmaceutically acceptable carriers, excipients, penetration enhancers, and/or adjuncts. Choosing the combination of pharmaceutically acceptable salts, carriers, excipients, penetration enhancers, and/or adjuncts in the composition depends in part upon the mode of administration and guidelines are known in the art.

Formulations may be administered to a subject or delivered to the biological sample conveniently in unit dosage form, which are prepared according to conventional techniques known in the pharmaceutical industry. In general terms, such techniques include bringing the test molecule into association with pharmaceutical carrier(s) and/or excipient(s) in liquid form or finely divided solid form, or both, and then shaping the product if required. The test molecule compositions may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

### EXAMPLES

The invention is further illustrated by the following examples, which should not be construed as limiting.

### Example 1

### Cell-Based Screens for Identifying Orally Active Alternatives to Erythropoietin

1. Manufacturing Bindingzyme Plates for Comparing Biological Samples

Bindingzymes are produced for known, suspected, hypothesized, or projected protein tyrosine phosphatases, protein serine/threonine phosphatases, and dual specificity or multispecificity protein phosphatases. These bindingzymes are used to bind phosphoproteins, the effector molecules in signal transduction. Each is expressed as an MBP fusion protein and each is affixed to a specific well of a Pierce Reacti-Bind™ Dextrin Coated Microwell Plate (as many wells and plates are used as needed to account for each bindingzyme). Duplicate plates and wells are generated to compare biological samples.

2. Preparation of Samples

Two identical cell cultures are used. One is exposed to erythropoietin (+EPO); the other is not (−EPO). Each culture is lysed, an aliquot of the +EPO sample is applied to one set of bindingzyme wells, and an aliquot of the −EPO sample is applied to a duplicate set of bindingzyme wells. Binding of peptides to the attached bindingzymes is allowed to occur at 25° C. for 20 minutes. All wells are washed with 1×PBS followed by washing with 25 mM NH₄HCO₃ buffer (pH 8.0). 10 μl of 25 mM NH₄HCO₃ buffer (pH 8.0) containing 2 units of calf intestine alkaline phosphatase is added to each well and incubated covered at 37° C. for 2 hours. Plates are centrifuged to collect sample at the bottom of each well. Approximately 15 nl of each sample is applied to a MALDI matrix spot of alpha-cyano-4-hydroxy-cinnamic acid, dried, and examined by MALDI-TOF mass spectrometry.

3. Configuring a High-throughput Screen

When results are compared between two samples, bindingzymes that detect a polypeptide variance between two samples (herein referred to as informative bindingzymes) are combined and saturating amounts applied to each and every well of Pierce Reacti-Bind™ Dextrin Coated Microwell Plates and excess amounts washed away. These plates of informative bindingzymes are then used to profile lysates from cultured cells that were treated with small molecules in order to find compounds that cause polypeptide variances from cultured cells not treated with EPO to partially or completely resemble polypeptide variances from cultured cells treated with EPO. Processing of the cell cultures is carried out as described above. Drug candidates then are examined in further assays involving cultured cells and animal models to identify orally active alternatives to erythropoietin.

### Example 2

### Cell-Based Screen for Identifying Orally Active Antagonists to TNF-alpha

Control Cell Cultures (without TNF-alpha) and Experimental Cell Cultures (with TNF-alpha) are analyzed as described in Example 1 using the panel of phoshphatase-derived bindingzymes. Informative bindingzymes then are combined into a single assay, or sometimes two or more assays. In contrast to the high-throughput screen in Example 1, however, cell cultures are pre-treated with chemical compounds, TNF-alpha is then added to each cell culture, and the cultures profiled. Cultures that resemble either fully or partially the control cultures, i.e., those that do not show the full effects of exposure to TNF-alpha, are cultures that were blocked by a chemical compound from responding fully to TNF-alpha treatment. The small number of compounds that meet these criteria are examined in detail using cultured cells and animal models.

### Example 3

### Cell-Based Screen for Identifying Antineoplastic Agents and Antimetastasis Agents

Using the panel of phoshphatase-derived bindingzymes described in Example 1, normal cell lines can be compared to their cancer counterparts. Highly metastatic cell lines can be compared to counterparts with low metastatic potential, as well as to normal cell lines. Informative bindingzymes (sometimes with selected non-informative bindingzymes) are then configured into a high-throughput screen. Test molecules are added to each biological sample and potential therapeutics are identified as test molecules that shift the abnormal phosphoprotein profile toward that of the normal counterpart. Therapeutic candidates then are examined in subsequent assays, such as assays involving cultured cells and animal models known in the art, for example.

### Example 4

### Cell-Based Screen for Assessing Specificity

Bindingzymes that do not detect variances between two samples (herein referred to as non-informative bindingzymes) can be very valuable in the subsequent development of drug candidates by assessing specificity. Non-informative bindingzymes are identified from the initial comparison of +EPO and −EPO cell cultures in Example 1. To best mimic the affects of EPO a drug candidate should not alter the pattern of phosphoproteins bound by these non-informative bindingzymes. Drug candidates can be ranked in terms of specificity by treating individual cell cultures with each of the drug candidates and comparing them to a control culture (i.e., a −EPO culture) using non-informative bindingzymes the more phosphoprotein variances detected using non-informative bindingzymes, the less the specificity. This resulting data is used in guiding medicinal chemistry efforts (e.g., modification of functional groups in a lead compound) for developing therapeutics more specific than those identified in initial screens.

### Example 5

### Cell-Based Screen for Assessing Toxicity

Application of the phosphatase-derived bindingzyme panel is used to assess toxicity of drug candidates. Cultured normal, primary human hepatocytes (liver cells) are profiled using the bindingzyme panel with and without known hepatotoxins. Drug candidates are likewise profiled and compared to the profiles of normal hepatocytes and the profiles from those treated with the known hepatotoxic agents. The drug candidates are ranked according to the likelihood that they cause hepatotoxicity based on the similarity to the profiles of known hepatotoxins. This screen is an important tool for reducing the toxicity of lead compounds identified in initial screens.

### Example 6

### Protein Phosphatase-based Screens for Identifying Drug Candidates

In some cases, high-throughput in vitro enzyme screens are preferable to cell-based screens. When it is probable that inhibition of a protein phosphatase will yield a desired therapeutic result, protein phosphatase assays may be utilized after initial profiling assays have been carried out (e.g., +/−EPO profiles of Example 1) in place of cell-based assays. For phosphopeptide profiles that change with +/−EPO, the identity of the peptides is determined by LC/MS/MS using samples not treated with alkaline phosphatase (i.e., the specific phosphate groups are retained prior to MS analysis in order to determine which amino acid residue(s) are phosphorylated). The phosphopeptides then are synthesized based upon the amino acid sequence deduced by the MS analysis, and the synthesized peptides are tested against the catalytically active phosphatase from which the informative bindingzyme was derived. MALDI-TOF MS is utilized to score the assay, since removal of a phosphate yields a readily scored shift in mass. The phosphatase assays (catalytically active phosphatases plus corresponding validated substrates) then are combined, which is possible because of the resolving power of the MALDI-TOF MS. A high-throughput screen of a chemical compound library is then conducted to identify protein phosphatase modulators by determining which compounds modulate phosphatase activity.

### Example 7

### Protein Kinase-based Screens for Identifying Drug Candidates

When it is probable that inhibition of a protein kinase will yield a desired therapeutic result, protein kinase assays may be utilized after initial profiling assays have been carried out (e.g., +/−TNF-alpha profiles of Example 2) in place of cell-based assays. For phosphopeptide profiles that change +/−TNF-alpha, the identity of the peptides that differ is determined by LC/MS/MS using samples not treated with alkaline phosphatase (i.e., the specific phosphate groups are retained prior to MS analysis in order to determine which amino acid residue(s) are phosphorylated). Non-phosphorylated peptides then are synthesized based upon the amino acid sequence deduced by the MS analysis, and the synthesized peptides are tested against a panel of known protein kinases. MALDI-TOF MS is used to score the assay, since addition of a phosphate yields a readily scored shift in mass. Protein kinase assays are combined (catalytically active protein kinases plus corresponding validated substrates), which is possible because of the resolving power of the MALDI-TOF MS. A high-throughput screen of a chemical compound library then is conducted to identify protein kinase modulators by determining which compounds modulate protein kinase activity.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications and patent documents cited herein are incorporated herein by reference as if each such publication or document was specifically and individually indicated to be incorporated herein by reference.

What is claimed is:

1. A method for identifying a molecule that reduces a polypeptide variance between two biological samples, which comprises:
contacting a first biological sample with one or more inactivated enzymes in a first system;
contacting a second biological sample with the one or more inactivated enzymes and one or more test molecules in a second system, wherein the one or more inactivated enzymes are capable of binding to a native polypeptide substrate or a fragment thereof and are catalytically defective, and wherein one or more of the inactivated enzymes are capable of detecting the presence of a polypeptide variance between the two biological samples;
detecting a signal corresponding to a polypeptide bound to the one or more inactivated enzymes in the second system and comparing the signal with a corresponding signal in the first system, whereby a test molecule that reduces the difference between the signals relative to the difference between the signals in the absence of the test compound is identified as a molecule that modulates a polypeptide variance between two biological samples.

2. The method of claim 1, wherein the molecule that modulates a polypeptide variance negates the difference between the signal from the first system and the signal from the second system.

3. The method of claim 1, wherein the inactivated enzyme is capable of binding to a binding site on the native polypeptide substrate or fragment thereof that comprises a modification capable of being added to the polypeptide by a native post-translational modification process.

4. The method of claim 3, wherein the modification is a phosphate moiety, ubiquitin moiety or acetyl moiety.

5. The method of claim 1, wherein the inactivated enzyme is a modified protein phosphatase, a modified deubiquitinase, a modified deacetylase, or a functional fragment thereof.

6. The method of claim 1, wherein the first system and/or second system comprise one or more inactivated enzymes incapable of detecting the presence of a polypeptide variance between the two biological samples in the absence of the one or more test molecules.

7. The method of claim 1, wherein a system is a well in a microtiter plate.

8. The method of claim 1, wherein one biological sample is contacted with one or more substances that are not contacted with the other biological sample.

9. The method of claim 8, wherein the one or more substances are selected from the group consisting of exogenous erythropoietin, exogenous TNF-alpha, exogenous toxin, an exogenous antimetastatic molecule and an exogenous antineoplastic molecule.

10. The method of claim 1, wherein one biological sample comprises cancerous cells and the other biological sample does not.

11. The method of claim 1, wherein the signal is a mass spectrometric signal.

12. The method of claim 11, wherein the signal is a MALDI-TOF signal.

13. An array comprising two or more inactivated enzymes immobilized to a solid support, wherein each inactivated enzyme is capable of binding to a native polypeptide substrate or a fragment thereof and is catalytically defective.

14. The array of claim 13, wherein one or more of the inactivated enzymes are capable of binding to a binding site on the native polypeptide substrate or fragment thereof that comprises a modification capable of being added to the polypeptide by a native post-translational modification process.

15. The array of claim 13, wherein one or more of the inactivated enzymes are capable of detecting the presence of a polypeptide variance between two biological samples.

16. The array of claim 13, wherein one or more of the inactivated enzymes is a modified phosphatase, a modified deubiquitinase, a modified deacetylase, or a functional fragment thereof.

17. The array of claim 13, wherein the solid support is a microtiter plate.

18. The array of claim 17, wherein one or more wells in the microtiter plate comprise one or more inactivated enzymes capable of detecting the presence of a polypeptide variance between two biological samples.

19. The array of claim 17, wherein one or more wells in the microtiter plate comprise one or more inactivated enzymes not capable of detecting the presence of a polypeptide variance between two biological samples.

20. The array of claim 17, wherein one or more wells in the microtiter plate comprise one or more inactivated enzymes capable of detecting the presence of a polypeptide variance between two biological samples and one or more inactivated enzymes not capable of detecting the presence of a polypeptide variance between two biological samples.

21. The array of claim 13, which comprises five or more inactivated enzymes.

22. A system comprising the array of claim 13 and a mass spectrometer.

23. A method for constructing an array of inactivated enzymes, which comprises:
contacting a first biological sample with inactivated enzymes in a first system;
contacting a second biological sample with the inactivated enzymes in a second system, wherein the inactivated enzymes are capable of binding to a native polypeptide substrate or a fragment thereof and are catalytically defective;
detecting and comparing signals corresponding to polypeptides bound to the inactivated enzymes in the first system and signals corresponding to polypeptides bound to the inactivated enzymes in the second system;
identifying inactivated enzymes for which there is a difference between a signal in the first system and a signal in the second system as informative inactivated enzymes and identifying inactivated enzymes for which there is no detectable difference between the signals as uninformative inactivated enzymes; and
depositing one or more of the informative inactivated enzymes in an array.

24. The method of claim 23, wherein the array comprises five or more inactivated enzymes.

25. The method of claim 23, which further comprises depositing one or more uninformative inactivated enzymes in the array.

26. The method of claim 25, wherein the array comprises five or more inactivated enzymes.

27. The method of claim 23, which further comprises depositing uninformative inactivated enzymes in a separate array.

28. The method of claim 27, wherein the separate array comprises five or more inactivated enzymes.
